(19) United States
(12) Patent Application Publication
(10) Pub. No.: US 2025/0351853 A1
(43) Pub. Date: Nov. 20, 2025

(54) FOOD COMPOSITION COMPRISING EQUOL AND PRODUCTION METHOD THEREFOR

(71) Applicant: Daicel Corporation, Osaka-shi, Osaka (JP)

(72) Inventor: Kazuya MITSUHASHI, Tokyo (JP)

(73) Assignee: Daicel Corporation, Osaka-shi, Osaka (JP)

(21) Appl. No.: 18/867,148

(22) PCT Filed: May 18, 2023
(86) PCT No.: PCT/JP2023/018548

§ 371 Date: Nov. 19, 2024

(30) Foreign Application Priority Data
May 19, 2022 (JP) 2022-082225

(51) Int. Cl. A23L 11/50; A23L 11/00; A23L 11/30; A23L 11/70; A61K 31/353; C12N 1/20

(52) U.S. Cl. CPC A23L 11/50; A23L 11/07; A23L 11/34; A23L 11/70; A61K 31/353; C12N 1/20

(57) ABSTRACT

The present disclosure provides an equol-containing food composition and a method for producing the same.

### TECHNICAL FIELD

The present invention relates to a food composition containing equol. The food composition is a liquid food composition having an adjusted pH or a dried product thereof.

The present invention also relates to a method for producing the food composition.

### BACKGROUND ART

Isoflavones, which are abundantly contained in leguminous plants such as soybeans and kudzu, are flavonoids that are a class of polyphenols and have an isoflavone backbone. Recent studies have revealed that isoflavones have a female hormonal action (estrogen) and an antioxidant action, and that the ingestion of isoflavones has a preventive effect on breast cancer, prostate cancer, osteoporosis, hypercholesterolemia, heart disease, menopausal issues, and the like.

Isoflavones are present in, for example, soybeans in the form of glycosides covalently bonded to sugars, such as daidzin, glycitin, and genistin, and are present in very small amounts in the form of aglycones. These glycosides may be further malonylated or acetylated and present in the malonylated or acetylated form. When these glycosides enter a human or animal body, the glycosides are converted to daidzein, glycitein, and genistein by the action of digestive enzymes or β-glucosidase, which is an enzyme produced by enterobacteria. Furthermore, it is known that daidzein is enzymatically converted to O-desmethylangolensin (O-DMA) or equol via dihydrodaidzein by the action of enterobacteria.

Equol is known to have the highest estrogenic activity among these metabolites. However, in humans, there are individual differences in the metabolism of isoflavones, and it is clear that few people possess enterobacteria having the ability to ferment daidzein to produce equol as described above. The prevalence rate of such enterobacteria is approximately 50% among Japanese people and approximately 30% among European and American populations. People who do not possess equol-producing bacteria cannot produce equol in their bodies even with the consumption of leguminous food such as soybeans, and therefore this inability to produce equol is an issue to be addressed.

In order to solve this issue, attempts have been made to produce equol in vitro using anaerobic microorganisms such as lactic acid bacteria (Patent Documents 1 to 4), and methods for efficiently producing equol and/or efficiently recovering the produced equol have been sought.

Patent Document 5 aims to provide an equol-producing microorganism-containing composition containing an equol-producing microorganism that maintains an equol-producing capability in a viable state and can stably maintain the equol-producing capability even after storage. Patent Document 5 also indicates that a pH adjuster may be added for pH control but does not disclose any specific pH adjuster. Patent Document 5 also indicates that the pH was adjusted in an anaerobic fermentation process, and that the preferable pH was 4.6 or greater.

### CITATION LIST

### Patent Document

Patent Document 1: JP 2006-204296 A
Patent Document 2: JP 2006-504409 T
Patent Document 3: JP 2008-61584 A
Patent Document 4: JP 2010-104241 A
Patent Document 5: JP 5769419 B

### SUMMARY OF INVENTION

### Technical Problem

Accordingly, an object of the present invention is to provide an equol-containing food composition that can further prevent microbial contamination and a method for producing the same.

In addition to the above object, another object of the present invention is to provide a method for producing the equol-containing food composition, the production method enabling efficient recovery of produced equol.

### Solution to Problem

To solve the above issues, the inventor of the present invention discovered the following inventions.
<1> A food composition containing equol, in which the food composition is a liquid food composition having an adjusted pH or a dried product thereof.
<2 The food composition according to <1>, wherein, desirably, the pH is adjusted to from 3 to 5, and preferably from 3 to 4.
<3> The food composition according to <2>, in which, desirably, the pH is adjusted using one acid selected from the group consisting of an organic acid and an inorganic acid,
the organic acid is at least one selected from the group consisting of carbonic acid, hydrogen carbonate, citric acid, succinic acid, fumaric acid, lactic acid, gluconic acid, acetic acid, malic acid, ascorbic acid, and benzoic acid, and
the inorganic acid is at least one selected from the group consisting of hydrochloric acid, sulfuric acid, and phosphoric acid.
<4> The food composition according to <2>, desirably further including at least one inorganic acid selected from the group consisting of hydrochloric acid, sulfuric acid, and phosphoric acid.
<5> The food composition according to <1>, in which, desirably, the pH is adjusted to from 7 to 11, and preferably from 10 to 11.
<6> The food composition according to <5>, in which, desirably, the pH is adjusted using one hydroxide selected from the group consisting of an alkali metal hydroxide and an alkaline earth metal hydroxide.
<7> The food composition according to <5>, in which, desirably, the pH is adjusted using one selected from the group consisting of sodium hydroxide, potassium hydroxide, calcium hydroxide, calcium oxide, and magnesium hydroxide.
<8> A liquid food composition production method for producing a pH-adjusted liquid food composition containing equol, the method including:
(A) culturing at least one equol raw material selected from the group consisting of daidzein glycoside, daidzein, and dihydrodaidzein using a microorganism that anabolizes the at least one equol raw material to produce equol; and
(B) adjusting a pH by adding a pH adjuster to an equol-containing culture solution produced in the culturing.
<9> A powdered food composition production method for producing a pH-adjusted powdered food composition containing equol, the method including:
(A) culturing at least one equol raw material selected from the group consisting of daidzein glycoside, daidzein, and dihydrodaidzein using a microorganism that anabolizes the at least one equol raw material to produce equol;
(B) adjusting a pH by adding a pH adjuster to an equol-containing culture solution produced in the culturing; and
(C) drying a liquid produced in the adjusting of the pH.
<10> The method according to <8> or <9>, in which, desirably, the pH is adjusted to from 3 to 5, and preferably from 3 to 4.
<11> The method according to <10>, in which, desirably,
the pH adjuster includes one selected from the group consisting of an organic acid and an inorganic acid,
the organic acid is at least one selected from the group consisting of carbonic acid, hydrogen carbonate, citric acid, succinic acid, fumaric acid, lactic acid, gluconic acid, acetic acid, malic acid, ascorbic acid, and benzoic acid, and
the inorganic acid is at least one selected from the group consisting of hydrochloric acid, sulfuric acid, and phosphoric acid.
<12> The method according to <10>, in which, desirably, the pH adjuster includes at least one inorganic acid selected from the group consisting of hydrochloric acid, sulfuric acid, and phosphoric acid.
<13> The method according to <8> or <9>, in which, desirably, the pH is adjusted to from 7 to 11, and preferably from 10 to 11.
<14> The method according to <13>, in which the pH adjuster desirably includes one selected from the group consisting of an alkali metal hydroxide and an alkaline earth metal hydroxide.
<15> The method according to <13>, in which the pH adjuster is desirably one selected from the group consisting of sodium hydroxide, potassium hydroxide, calcium hydroxide, calcium oxide, and magnesium hydroxide.

### Advantageous Effects of Invention

According to the present invention, an equol-containing food composition that can further prevent microbial contamination, and a method for producing the same can be provided.

Moreover, according to the present invention, in addition to the abovementioned effect, a method for producing the equol-containing food composition with efficient recovery of the produced equol can be provided.

### DESCRIPTION OF EMBODIMENTS

The present application provides a method for producing a liquid food composition and a method for producing a powdered food composition, and also provides a liquid food composition and a dried product thereof, that is, a powdered food composition.

Hereinafter, the method for producing a liquid food composition and the method for producing a powdered food composition will be described, after which the liquid food composition and the dried product thereof, that is, the powdered food composition, will be described.

### Liquid Food Composition Production Method

A liquid food composition production method according to an embodiment of the present invention includes the following steps:
(A) culturing at least one equol raw material selected from the group consisting of daidzein glycoside, daidzein, and dihydrodaidzein using a microorganism that anabolizes the at least one equol raw material to produce equol; and
(B) adjusting a pH by adding a pH adjuster to an equol-containing culture solution produced in the culturing step,
and thereby a pH-adjusted liquid food composition containing equol is produced.

### Step (A)

In step (A), at least one equol raw material selected from the group consisting of daidzein glycoside, daidzein and dihydrodaidzein using a microorganism that anabolizes the equol raw material is cultured to produce equol.

Conditions and the like in the culturing step are not particularly limited as long as equol can be produced. For example, typically known conditions can be used, but the conditions are not limited thereto.

### Equol Raw Material

The equol raw material used in the method according to an embodiment of the present invention may be in any form as long as the material can literally be used as a raw material for equol.

The equol raw material may be in any form as long as the equol contains at least one selected from the group consisting of daidzein glycoside, daidzein, and dihydrodaidzein. Examples of the equol raw material include daidzein glycoside itself, daidzein itself, and dihydrodaidzein itself, as well as raw materials containing these, such as soybeans, processed soybeans, soybean hypocotyls, and processed soy bean hypocotyls (e.g., soybean extracts, soybean hypocotyl extracts, and purified soy bean hypocotyl extracts), and specifically, commercially available isoflavones may be used.

### Microorganism

The method according to an embodiment of the present invention uses a microorganism that has the ability to anabolize an equol raw material to produce equol. The “ability to anabolize an equol raw material to produce equol” may be simply referred to herein as an “equol-producing capability”.

The microorganism having an equol-producing capability and used in the method according to an embodiment of the present invention is not particularly limited as long as the microorganism is one having the ability to produce equol from the above-mentioned equol raw material.

Note that the equol raw material is determined by the relationship with the “equol-producing capability” of the microorganism. For example, when a certain microorganism A does not have an “equol-producing capability” with regard to daidzein glycoside but has an “equol-producing capability” with regard to daidzein, “daidzein” is selected as the equol raw material for the microorganism A. In this case, step (A) may be preceded by a step of converting daidzein glycoside into daidzein. Also, for example, when a certain microorganism B does not have an “equol-producing capability” for daidzein glycosides and daidzein, but has an “equol-producing capability” for dihydrodaidzein, “dihydrodaidzein” is selected as the equol raw material for the microorganism B. In this case, step (A) may be preceded by a step of converting daidzein glycoside into daidzein and further converting daidzein into dihydrodaidzein.

Examples of the microorganism include anaerobic microorganisms. The anaerobic microorganisms can produce equol at a temperature of, for example, around 37° C. (e.g., from 30 to 42° C.).

The equol-producing capability can be confirmed by quantitatively determining the daidzein, dihydrodaidzein, equol, and the like in the culture. A person skilled in the art can carry out these quantitative determinations on the basis of the descriptions of, for example, WO 2012/033150, JP 2012-135217 A, JP 2012-135218 A, and JP 2012-135219 A. An example of these quantitative determination methods is described below.

For example, ethyl acetate is added to a culture solution, the mixture is vigorously stirred and then centrifuged, and the ethyl acetate layer is extracted. The same operation can be carried out several times on the same culture solution as necessary, and the extracted ethyl acetate layers can be combined to produce a liquid extract of equol. The liquid extract is concentrated and dried under reduced pressure using an evaporator, and then dissolved in methanol. The resulting solution is filtered using a membrane such as a polytetrafluoroethylene (PTFE) membrane to remove insoluble matter, and the resulting product can be used as a sample for high performance liquid chromatography. Examples of the conditions for high performance liquid chromatography include, but are not limited to, the following.

### Conditions for High Performance Liquid Chromatography

Column: Phenomenex Luna 5uC18, 2.0 mm×150 mm (Shimadzu Glc Ltd.)
Mobile phase: water/methanol [55:45, v/v]
Flow rate: 0.2 mL/min
Column temperature: 40° C.
Detection: UV 280 nm
Retention time: 13.8 minutes for dihydrodaidzein, 19.6 minutes for daidzein, 22.5 minutes for glycitein, 25.6 minutes for equol, 35.0 minutes for genistein

Examples of microorganisms having the ability to produce equol include, but are not limited to, microorganisms classified into the following genera.
Genus Adlercreutzia
Genus Bacteroides
Genus Bifidobacterium
Genus Clostridium
Genus Eggerthella
Genus Enterococcus
Genus Enterorhabdus
Genus Eubacterium
Genus Finegoldia
Genus Lactobacillus
Genus Lactococcus
Genus Paraeggerthella
Genus Pediococcus
Genus Proteus
Genus Sharpea
Genus Slackia
Genus Streptococcus
Genus Veillonella

Specific examples of microorganisms having the ability to produce equol include, but are not limited to, the following microorganisms.
Adlercreutzia equolifaciens subsp. celatus
Adlercreutzia equolifaciens subsp. equolifaciens
Bacteroides ovatus
Bifidobacterium breve
Bifidobacterium longum
Clostridium sp.
Eggerthella sp.
Enterococcus faecalis
Enterococcus faecium
Enterorhabdus mucosicola
Eubacterium sp.
Finegoldia magna
Lactobacillus fermentum
Lactobacillus mucosae
Lactobacillus paracasei
Lactobacillus plantarum
Lactobacillus rhamnosus
Lactobacillus sp.
Lactococcus garvieae
Lactococcus sp.
Paraeggerthella sp.
Pediococcus pentosaceus
Proteus mirabilis
Sharpea azabuensis
Slackia equolifaciens
Slackia isoflavoniconvertens
Slackia sp.
Streptococcus constellatus
Streptococcus intermedius
Veillonella sp.

Examples of the above-described microorganisms include microorganisms classified into the Eggerthellaceae family, microorganisms classified into the Bifidobacteriaceae family, microorganisms classified into the Clostridiaceae family, microorganisms classified into the Coriobacteriaceae family, microorganisms classified into the Enterococcaceae family, microorganisms classified into the Eubacteriaceae family, microorganisms classified into the Morganellaceae family, microorganisms classified into the Peptoniphilaceae family, microorganisms classified into the Lactobacillus family, microorganisms classified into the Streptococcaceae family, microorganisms classified into the Veillonellaceae family, and related microorganisms thereof. Preferable microorganisms are those classified into the genus Adlercreutzia, the genus Bacteroides, the genus Bifidobacterium, the genus Clostridium, the genus Coriobacterium, the genus Eggerthella, the genus Enterococcus, the genus Eubacterium, the genus Finegoldia, the genus Lactobacillus, the genus Lactococcus, the genus Paraeggerthella, the genus Pediococcus, the genus Proteus, the genus Sharpea, the genus Slackia, the genus Streptococcus, and the genus Veillonella, or related microorganisms thereof. More preferable microorganisms are Adlercreutzia equolifaciens subsp. celatus, Adlercreutzia equolifaciens subsp. equolifaciens, Bacteroides ovatus, Bifidobacterium breve, Bifidobacterium longum, Clostridium sp., Eggerthella sp., Enterococcus faecalis, Enterococcus faecium, Enterorhabdus mucosicola, Eubacterium sp., Finegoldia magna, Lactobacillus fermentum, Lactobacillus intestinalis, Lactobacillus mucosae, Lactobacillus paracasei, Lactobacillus plantarum, Lactobacillus rhamnosus, Lactobacillus sp., Lactococcus garvieae, Lactococcus sp., Paraeggerthella sp., Pediococcus pentosaceus, Proteus mirabilis, Sharpea azabuensis, Slackia equolifaciens, Slackia isoflavoniconvertens, Slackia sp., Streptococcus constellatus, Streptococcus intermedius, and the Veillonella sp.

Among the above-described microorganisms, particular examples of more preferable anaerobic microorganisms include any of the microorganisms described below or related bacteria having the same species properties as these microorganisms.
Adlercreutzia equolifaciens subsp. celatus DSM 18785 strain
Adlercreutzia equolifaciens subsp. equolifaciens DSM 19450 strain
Bacteroides ovatus E-23-15 strain
Bifidobacterium breve ATCC 15700 strain
Bifidobacterium longum BB536 strain
Clostridium sp. HGH136 strain
Eggerthella sp. Julong 732 strain
Eggerthella sp. YY7918 strain
Eggerthella sp. D1 strain
Enterococcus faecalis INIA P333 strain
Enterococcus faecium EPI1 strain
Enterorhabdus mucosicola Mt1B8 strain
Eubacterium sp. D2 strain
Finegoldia magna EPI3 strain
Lactobacillus fermentum DPPMA114 strain
Lactobacillus intestinalis KTCT13676BP strain
Lactobacillus mucosae EPI2 strain
Lactobacillus paracasei JS1 strain
Lactobacillus plantarum DPPMA24W strain
Lactobacillus plantarum DPPMASL33 strain
Lactobacillus rhamnosus DPPMAAZI strain
Lactobacillus rhamnosus INIA P540 strain
Lactobacillus sp. Niu-O16 strain
Lactococcus garvieae 20-92 strain
Paraeggerthella sp. SNR40-432 strain
Pediococcus pentosaceus CS1 strain
Proteus mirabilis LH-52 strain
Sharpea azabuensis ST18 strain
Slackia equolifaciens strain DSM 24851 strain
Slackia isoflavoniconvertens DSM 22006 strain
Slackia sp. FJK1 strain
Slackia sp. NATTS strain
Slackia sp. YIT11861 strain
Slackia sp. TM-30 strain
Streptococcus constellatus E-23-17 strain
Streptococcus intermedius A6G-225 strain
Veillonella sp. EP strain

Note that the abovementioned anaerobic microorganisms are available from the depository indicated by the deposit number. Each accession number indicates that the anaerobic microorganism is deposited in one of the following depositories.
FERM: International Patent Organism Depositary (IPOD) http://unit.aist.go.jp/pod/ci/index.html
DSM: German Collection of Microorganisms and Cell Cultures (DSMZ) http://www.dsmz.de/
KCCM: Korean Culture Center of Microorganisms

In the present invention, the anaerobic microorganism capable of producing equol is cultured under conditions suitable for the production of equol. In the present invention, the conditions suitable for the production of equol refer to conditions under which the survival and activity of the anaerobic microorganism having equol-production activity are maintained. More specifically, the conditions thereof refer to conditions under which the gas phase conditions (anaerobic conditions) in which anaerobic microorganisms can survive are maintained, and nutrients for supporting the activity and growth of the anaerobic microorganisms are provided. Various culture medium compositions suitable for the survival of the anaerobic microorganisms are known. Therefore, a person skilled in the art can select an appropriate culture medium composition for an above-described anaerobic microorganism having the ability to produce equol. For example, a BHI culture medium available from Difco Laboratories Inc. or a culture medium used in the examples can be used.

A water-soluble organic material can be added as a carbon source to the culture medium used in the present invention. Examples of the water-soluble organic material include, but are not limited to, the following compounds:
saccharides such as sorbose, fructose, and glucose; alcohols such as methanol; and organic acids such as valeric acid, butyric acid, propionic acid, acetic acid, and formic acid, or salts thereof.

The concentration of the organic material added to the culture medium as a carbon source can be adjusted, as appropriate, to efficiently grow anaerobic microorganisms in the culture medium.

A nitrogen source can be added to the culture medium. Various nitrogen compounds that can be ordinarily used in fermentation can be used as the nitrogen source in the present invention. Preferred inorganic nitrogen sources include ammonium salts and nitrates. More preferable inorganic nitrogen sources include ammonium sulfate, ammonium chloride, ammonium phosphate, ammonium hydrogen phosphate, potassium nitrate, and sodium nitrate. Meanwhile, examples of preferred organic nitrogen sources include amino acids, yeast extracts, peptones, meat extracts, liver extracts, and digested serum powder. Examples of more preferred organic nitrogen sources include arginine, cysteine, cystine, citrulline, lysine, yeast extracts, and peptones.

Furthermore, other organic materials or inorganic materials suited for the production of equol can also be added to the culture medium in addition to the carbon source and the nitrogen source. For example, in some cases, the growth and activity of anaerobic microorganisms can be enhanced by adding cofactors such as vitamins or inorganic compounds such as various salts to the culture medium. Examples of inorganic compounds, vitamins, and plant- and animal-derived cofactors for microbial growth include the following.

| Inorganic Compounds | vitamins |
|---|---|
| Potassium dihydrogen phosphate | biotin |
| Magnesium sulfate | folic acid |
| Manganese sulfate | pyridoxine |
| Sodium chloride | thiamine |
| Cobalt(II) chloride | riboflavin |
| Calcium chloride | nicotinic acid |
| Zinc sulfate | pantothenic acid |
| Copper sulfate | vitamin B12 |
| Alum | thioctic acid |
| Sodium molybdate | p-aminobenzoic acid |
| Potassium chloride | |
| Boric acid and the like | |
| Nickel(II) chloride | |
| Sodium tungstate | |
| Sodium selenate | |
| Ammonium iron(II) sulfate | |

A typically known technique can be used as the method for producing a culture solution by adding these inorganic compounds, vitamins, or growth cofactors. The culture medium can be a liquid, a semi-solid or a solid. In the present invention, the preferred form of the culture medium is a liquid culture medium.

The culture medium used in the present invention may contain dextrins. When an anaerobic microorganism is cultured in a culture medium containing dextrins, a liquid containing equol and dextrins can be prepared without bringing the dextrins into contact with the culture after the culturing.

The dextrins can be added to the culture medium before or during the culturing of the microorganisms.

The culture medium used in the present invention may contain an antifoaming agent, preferably soybean oil, and more preferably soybean oil containing vitamin E.

In the method of the present application, microorganisms, and particularly anaerobic microorganisms can be cultured according to a known method of culturing microorganisms. In industrial production, a continuous cultivation system (continuous fermentation system) that can continuously feed the culture medium and a gaseous substrate and is provided with a mechanism for recovering the culture can also be used.

When anaerobic microorganisms are used in the method according to an embodiment of the present invention, it is preferable to prevent oxygen from entering the fermenter. As the fermenter, a commonly used fermenter can be used as is. An anaerobic atmosphere can be created by replacing oxygen that mixes into the fermenter with an inert gas such as nitrogen.

In step (A) according to an embodiment of the present invention, the gas phase is preferably composed of one or more types of gases including hydrogen. The gas constituting the gas phase is not particularly limited as long as the gas is composed of one or more types of gases including hydrogen, but the gas phase preferably contains hydrogen and one or more types of gases other than hydrogen. Examples of the gas other than hydrogen include, but are not limited to, carbon dioxide, nitrogen, and carbon monoxide.

The hydrogen concentration of the abovementioned gas is not particularly limited, and may be, for example, 30% or less, 10% or less, or 4% or less.

Note that step (A) in the present invention may be carried out in a closed system such as a bottle or a test tube tightly sealed with a rubber stopper without aeration.

In order to efficiently recover equol, the aeration amount of the mixed gas constituting the gas phase into the culture vessel can be set to a level from 0.001 to 2.0 V/V/M gas amount/liquid amount/min, and for example, can be set to a level from 0.01 to 2.0 V/V/M gas amount/liquid amount/min, but the aeration amount is not limited thereto.

Depending on the shape of the culture vessel, a stirrer or the like can be used to sufficiently stir the culture medium. The production efficiency of equol can be optimized by stirring the culture in the culture vessel to thereby increase the opportunities for contact of the components of the culture medium and the gaseous substrate with anaerobic microorganisms. The gaseous substrate can also be supplied as nanobubbles.

In the present invention, microorganisms can also be cultured at normal pressure, but when microorganisms are to be cultured under pressure, the pressurization condition for cultivation of the microorganisms is not particularly limited as long as the condition allows for growth. Preferable pressurization conditions include, but are not limited to, a range of 0.2 MPa or less, and for example, a range of from 0.02 to 0.2 MPa.

Further, the temperature of the culture vessel is not particularly limited, but is preferably from 30° C. to 40° C., and more preferably from 33° C. to 38° C. from the viewpoint of increasing the production amount of equol.

The culturing time can be appropriately set according to the production amount of equol, the remaining amount of isoflavones, and the like. The culturing time is, for example, from 8 to 120 hours, preferably from 12 to 72 hours, and particularly preferably from 16 to 60 hours, but is not limited thereto.

### Step (B)

In step (B), the pH is adjusted by adding a pH adjuster to the equol-containing culture solution produced in step (A), that is, the culturing step.

Through the implementation of step (B), the pH of the culture solution may be adjusted to the acidic range, and specifically, the pH may be adjusted to from 3 to 5, and preferably from 3 to 4. By adjusting the pH of the culture solution to the acidic range in this manner, and more specifically, by adjusting the pH to from 3 to 5, and particularly from 3 to 4, the microorganisms used in step (A) can be easily separated, and consequently the recovery efficiency of equol can be enhanced.

Through step (B), the pH of the culture solution may be adjusted to the alkaline range, and specifically, the pH may be adjusted to from 7 to 11, and preferably from 10 to 11. By adjusting the pH of the culture solution to the alkaline range in this manner, and more specifically, by adjusting the pH to from 7 to 11, and particularly from 10 to 11, the microorganisms used in step (A) can be easily dissolved, and consequently the recovery efficiency of equol can be enhanced.

When the pH is to be adjusted to the acidic range in step (B), the pH adjuster preferably includes at least one acid selected from the group consisting of organic acids and inorganic acids.

Examples of the organic acids may include, but are not limited to, carbonic acid, hydrogen carbonate, citric acid, succinic acid, fumaric acid, lactic acid, gluconic acid, acetic acid, malic acid, ascorbic acid and benzoic acid.

Examples of the inorganic acids may include, but are not limited to, hydrochloric acid, sulfuric acid, and phosphoric acid.

When the pH is to be adjusted to the alkaline range in step (B), the pH adjuster preferably includes one hydroxide selected from the group consisting of alkali metal hydroxides and alkaline earth metal hydroxides.

Specific examples of the pH adjuster for adjusting the pH to the alkaline range include, but are not limited to, sodium hydroxide, potassium hydroxide, calcium hydroxide, calcium oxide, and magnesium hydroxide.

While the liquid food composition can be produced by the above steps (A) and (B), the method according to an embodiment of the present invention for producing the liquid food composition may include steps other than the above steps (A) and (B). Examples of such other steps include, but are not limited to, a step of removing microorganisms having the ability to produce equol, such as a centrifugation step and a membrane filtration step.

### Powdered Food Composition Production Method

The present application further provides a method for producing a powdered food composition.

The powdered food composition production method includes, after steps (A) and (B),
(C) drying the liquid obtained in the pH adjustment step (B),
and thereby, a pH-adjusted powdered food composition containing equol can be produced.

### Step (C)

In step (C), the liquid produced in the pH adjustment step (B) is dried.

The drying step (C) may be carried out by a typically known method. Examples thereof include, but are not limited to, a heated drying process, a spray drying process, a freeze drying process, fluidized bed drying, and fluidized layer drying.

The heated drying process can be carried out using, for example, a rotary drum dryer, the spray drying process can be carried out using, for example, a spray dryer, and the freeze drying process can be carried out using a freeze dryer. The drying method may use any dryer as long as the dryer can dry the liquid.

The product obtained through a drying process may be subjected to a grinding process as necessary.

The method according to an embodiment of the present invention may include steps in addition to the above steps (A), (B) and (C) described above.

For example, after step (B) and before step (C), the method may include a step of heating the pH-adjusted liquid produced in step (B), and a centrifugation step and/or a filtration step for removing an unnecessary solid from the obtained liquid after the heating step. Note that in step (B), when the pH is adjusted to the alkaline range, and in particular, when the pH is adjusted to from 10 to 11, the centrifugation step need not be included, which is preferable in terms of reducing the number of steps.

When a centrifugation step and/or a filtration step for removing an unnecessary solid content is included, a clear liquid is produced, and thus a liquid food composition suitable for serving as a beverage or the like is produced.

### Food Composition

The present application provides a food composition containing equol, the food composition being a liquid food composition having an adjusted pH or a dried product thereof.

The food composition according to an embodiment of the present invention can also be produced by the method described above, but is not limited thereto.

### Liquid Food Composition

When in the acidic range, the liquid food composition has a pH of from 3 to 5, and preferably from 3 to 4.

In addition, when the liquid food composition is in the alkaline range, the pH thereof is from 7 to 11, and preferably from 10 to 11.

### Dried Product of Liquid Food Composition

The present application also provides a dried product of the pH-adjusted liquid food composition containing equol.

In the dried product, the pH is defined as follows.

That is, when 50 g of the dried product is dissolved or suspended in 1 L of water, the pH is in the above-mentioned range, that is, when in the acidic range, the pH is from 3 to 5 and is preferably from 3 to 4, or when in the alkaline range, the pH is from 7 to 11 and is preferably from 10 to 11.

To measure the pH, a glass diaphragm electrode can be suitably used, but a simple method such as a pH test paper can also be used.

When the pH of the food composition according to an embodiment of the present invention is adjusted to the acidic range, the pH is preferably adjusted with one acid selected from the group consisting of organic acids and inorganic acids.

In this case, the food composition according to an embodiment of the present invention contains one type of acid selected from the group consisting of organic acids and inorganic acids.

Here, examples of the organic acids may include, but are not limited to, carbonic acid, hydrogen carbonate, citric acid, succinic acid, fumaric acid, lactic acid, gluconic acid, acetic acid, malic acid, ascorbic acid, and benzoic acid.

Examples of the inorganic acids include, but are not limited to, hydrochloric acid, sulfuric acid, and phosphoric acid.

When the pH of the food composition according to an embodiment of the present invention is adjusted to the alkaline range, the pH is preferably adjusted with one hydroxide selected from the group consisting of hydroxides of alkali metals and hydroxides of alkaline earth metals.

In this case, the food composition according to an embodiment of the present invention contains one hydroxide selected from the group consisting of hydroxides of alkali metals and hydroxides of alkaline earth metals.

Examples of the one type of hydroxide selected from the group consisting of hydroxides of alkali metals and hydroxides of alkaline earth metals include, but are not limited to, sodium hydroxide, potassium hydroxide, calcium hydroxide, calcium oxide, and magnesium hydroxide.

Hereinafter, the present invention will be described on the basis of examples, but the scope of the present invention is not limited to the following examples.

### EXAMPLES

### Example 1

### Preparation of Preculture Medium

Anaerobe Basal Broth (available from Thermo Fisher Scientific Inc, Catalog No. CM0957B) was dissolved in a predetermined amount of distilled water (ABB culture medium), and 1 L of the solution was dispensed into a 2 L pressurized fermenter. Subsequently, gas purge was carried out with nitrogen gas, and sterilization was carried out at 121° C. for 15 minutes.

### Preparation of Main Culture Medium

An enzyme-treated soybean germ extract (containing aglyconized isoflavones: daidzein, glycitein, and genistein) was added to an ABB culture medium containing β-cyclodextrin to achieve a final concentration of 6 g/L, and 1 L of the mixture was dispensed into a 2 L fermenter. Subsequently, gas purge was carried out with nitrogen gas, and sterilization was carried out at 121° C. for 15 minutes.

### Preculture

The preculture medium was inoculated with the Adlercreutzia equolifaciens DSM 19450 strain, after which culturing was carried out at 37° C. for 1 day while a hydrogen-containing anaerobic gas sterilized by a sterilization filter (pore size: 0.2 μm, material: polyvinylidene fluoride (PVDF)) was continuously supplied.

### Main Culture

A main culture medium was inoculated with the precultured strain, after which culturing was carried out at 37° C. for 2 days while a hydrogen-containing anaerobic gas sterilized by a sterilization filter (pore size: 0.2 μm, material: PVDF) was continuously supplied.

The production of equol was confirmed through high-performance liquid chromatography (HPLC) analysis.

### pH Adjustment Step

The main culture solution in which equol production was confirmed was collected in a glass container, the pH was adjusted, and the solution was sterilized through heating. For comparison, a sample was prepared in which sterilization by heating was carried out without adjusting the pH.
1) Citric acid was added to the main culture solution to reduce the pH to 3, 4, or 5, and sterilization by heating was carried out.
2) Hydrochloric acid was added to the main culture solution to reduce the pH to 3, 4, or 5, and sterilization by heating was carried out.
3) Sodium hydroxide was added to the main culture solution to increase the pH to 8, 9, 10, or 11, and sterilization by heating was carried out.

### Confirmation of Bacteriostatic Effect

The heat-sterilized liquid produced in the above pH adjustment step was allowed to stand as is at room temperature for one week. Subsequently, a portion of the room temperature liquid was applied to a standard agar medium at an equal amount each time, and cultured at 37° C. for 1 day, and the number of colonies was counted.

The results are indicated in Table 1.

From Table 1, it was confirmed that in the case in which the pH was not adjusted, the number of colonies was so large that the colonies could not be counted, whereas it was also confirmed that bacterial growth can be suppressed by adjusting the pH to the acidic range or the alkaline range through the pH adjustment step. It was found that microbial contamination can be prevented by providing the pH adjustment step according to an embodiment of the present invention and by using the pH-adjusted liquid food composition according to an embodiment of the present invention or a dried product thereof.

TABLE 1
Bacteria Count Measurement Results

| | pH | | | | | | | |
|---|---|---|---|---|---|---|---|---|
| | | | | 6.6 (no | | | | |
| pH adjuster | 3 | 4 | 5 | adjustment) | 8 | 9 | 10 | 11 |
| No adjustment | | | | *1 | | | | |
| Citric acid | 0 | 0 | 15 | | | | | |
| Hydrochloric acid | 0 | 0 | 0 | | | | | |
| NaOH | | | | | 350 | 0 | 0 | 0 |

*1: Too large to count
Note:
The numerical values in the table indicate the number of colonies.

### Example 2

The steps in Example 1 were carried out until the pH adjustment step, after which the following step was carried out.

### Centrifugal Precipitation Step

The heat-sterilized culture solution was inserted into a 2 mL Eppendorf tube, insoluble components were precipitated by centrifugation, the supernatant was removed, and the amount of precipitate was confirmed. At this time, the influence on precipitation was observed by changing the centrifugation conditions (rotational speed, time).

The results are listed in Table 2.

From Table 2, it was found that when the pH was adjusted to the acidic range, the amount of precipitation did not change even when the rotational speed of centrifugation was decreased, but when the pH was 7 or greater, the apparent amount of precipitation increased.

Thus, it was confirmed that adjusting the pH affects the separation characteristics in the centrifugation step.

TABLE 2
Amount of Precipitation (The numbers in the table are represented in mg.)

| | 12000 rpm × 2 min | | | | 8000 rpm × 2 min | | | |
|---|---|---|---|---|---|---|---|---|
| | No | Citric | Hydrochloric | | No | Citric | Hydrochloric | |
| pH | adjustment | acid | acid | NaOH | adjustment | acid | acid | NaOH |
| 3 | | 36 | 37 | | | 36 | 36 | |
| 4 | | 41 | 41 | | | 41 | 41 | |
| 5 | | 38 | 38 | | | 38 | 38 | |
| 6.6 No | 38 | | | | 48 | | | |
| adjustment | | | | | | | | |
| 8 | | | | 39 | | | | 49 |
| 9 | | | | 32 | | | | 44 |
| 10 | | | | 16 | | | | 26 |
| 11 | | | | 13 | | | | 26 |

### Example 3

The steps in Example 1 were carried out until the pH adjustment step, after which the following step was carried out.

### Centrifugal Precipitation Step

After the pH was adjusted, the heat-sterilized culture solution was inserted into a 40 mL centrifuge tube, insoluble components were precipitated using a centrifuge, and the supernatant was collected.

### Filtration Step

The supernatant collected by centrifugal precipitation was filtered through a microfiltration (MF) membrane made of PVDF (pore size: 0.2 μm), and the filtrate was collected. Equol and isoflavone in the filtrate were analyzed by the HPLC method.

The results thereof are presented in Table 3.

From Table 3, it was found that the isoflavone concentration was improved and the recovery rate of isoflavone in the filtrate was increased by adjusting the pH to the alkaline range in the pH adjustment step. Since isoflavones other than equol can also be expected to have physiological activity, an effect that cannot be achieved by equol alone can be expected by improving the isoflavone concentration.

TABLE 3
Filtrate Recovery Amount and Analysis Results

| | | Filtrate Recovery Rate | | |
|---|---|---|---|---|
| | | (Filtrate amount/amount | Equol | Isoflavone |
| pH adjuster | pH | of culture solution) | (g/L) | (g/L) |
| NaOH | 8 | 88.5% | 2.80 | 2.88 |
| | 9 | 89.0% | 2.71 | 2.88 |
| | 10 | 90.6% | 2.67 | 3.29 |
| | 11 | 90.9% | 2.58 | 3.35 |

### Example 4

The steps in Example 1 were carried out until the pH adjustment step, after which the following drying step was carried out.

In addition, the steps in Example 3 were carried out until the filtration step, after which the following drying step was carried out.

### Drying Step

The filtrate was subjected to freeze drying, and a powder was recovered.

### Confirmation of Bacteriostatic Effect

The recovered powder was spread on a petri dish and allowed to stand as is at room temperature for one week. Subsequently, a portion thereof was used to prepare an agar culture medium by the pour-in method using a standard agar medium, and then cultured at 37° C. for 1 day, and the number of colonies was counted.

No colonies were detected in any of the powders. Thus, it is conceivable that the bacteriostatic effect was maintained even after drying.

Note that Table 4 shows pH measurement results for liquids produced by dissolving or suspending 50 g of the recovered powder in 1 L of water or at a ratio equivalent thereto. From Table 4, it was confirmed that the pH (the “post-drying” value in Table 4) of the liquid obtained by re-dissolving the recovered powder was substantially the same as the pH (the “pre-drying” value in Table 4) used in the pH adjustment step of Example 1.

TABLE 4
pH Measurement Result of Liquid Produced
by Redissolving Recovered Powder

| | | | | | | | | |
|---|---|---|---|---|---|---|---|---|
| Pre-drying | 3 | 4 | 5 | 6.6 (no adjustment) | 8 | 9 | 10 | 11 |
| Post-drying | 3.1 | 4.2 | 5.1 | 6.7 | 8.2 | 9.0 | 9.9 | 10.8 |

### Example 5

The same procedure as in Example 1 was carried out with the exception that the Asaccharobacter celatus DSM 18785 strain was used instead of the Adlercreutzia equolifaciens DSM 19450 strain used in Example 1.

The results are presented in Table 5.

From Table 5, it was confirmed that, as in Example 1, in the case in which the pH was not adjusted, the number of colonies was so large that the colonies could not be counted, whereas bacterial growth was suppressed by adjusting the pH to the acidic range or the alkaline range through the pH adjustment step. It was found that microbial contamination can be prevented by providing the pH adjustment step according to an embodiment of the present invention and by using the pH-adjusted liquid food composition according to an embodiment of the present invention or a dried product thereof.

TABLE 5
Bacterial Count Measurement Results

| | pH | | | | | | | |
|---|---|---|---|---|---|---|---|---|
| | | | | 6.9 (no | | | | |
| pH adjuster | 3 | 4 | 5 | adjustment) | 8 | 9 | 10 | 11 |
| No adjustment | | | | *1 | | | | |
| Citric acid | 0 | 0 | 23 | | | | | |
| Hydrochloric acid | 0 | 0 | 0 | | | | | |
| NaOH | | | | | 290 | 0 | 0 | 0 |

*1: Too large to count
Note:
The numerical values in the table indicate the number of colonies.

### Example 6

The same procedure as in Example 3 was carried out with the exception that the Asaccharobacter celatus DSM 18785 strain was used instead of the Adlercreutzia equolifaciens DSM 19450 strain that was used in Example 3.

The results are presented in Table 6.

Table 6 shows that the isoflavone concentration was improved and the recovery rate of isoflavone in the filtrate was increased by adjusting the pH to the alkaline range in the pH adjustment step. Since isoflavones other than equol can also be expected to have physiological activity, an effect that cannot be achieved by equol alone can be expected by improving the isoflavone concentration.

TABLE 6
Filtrate Recovery Amount and Analysis Results

| | | Filtrate Recovery Rate | | |
|---|---|---|---|---|
| | | (Filtrate amount/amount | Equol | Isoflavone |
| pH adjuster | pH | of culture solution) | (g/L) | (g/L) |
| NaOH | 8 | 88.2% | 2.82 | 2.92 |
| | 9 | 88.9% | 2.74 | 2.85 |
| | 10 | 90.3% | 2.68 | 3.31 |
| | 11 | 90.4% | 2.45 | 3.31 |

### Example 7

### Preparation of Preculture Medium

Anaerobe Basal Broth (available from Thermo Fisher Scientific Inc, Catalog No. CM0957B) was dissolved in a predetermined amount of distilled water (ABB culture medium), and 1 L of the solution was dispensed into a 2 L pressurized fermenter. Subsequently, gas purge was carried out with nitrogen gas, and sterilization was carried out at 121° C. for 15 minutes.

### Preparation of Main Culture Medium

After the soybean germs were ground, tap water was added to achieve a concentration of ground soybean germ of 100 g/L, and 1 L of the mixture was dispensed into a 2 L fermenter. Subsequently, enzymes were added, and the mixture was stirred overnight at 50° C. to convert the isoflavone glycosides contained therein into aglycones, after which arginine was added to a concentration of 1 g/L, the gas was purged with nitrogen gas, and then sterilization was carried out at 121° C. for 15 minutes.

### Preculture

The preculture medium was inoculated with the Adlercreutzia equolifaciens DSM 19450 strain, after which culturing was carried out at 37° C. for 1 day while a hydrogen-containing anaerobic gas sterilized by a sterilization filter (pore size: 0.2 μm, material: polyvinylidene fluoride (PVDF)) was continuously supplied.

### Main Culture

A main Fculture medium was inoculated with the precultured strain, after which culturing was carried out at 37° C. for 2 days while a hydrogen-containing anaerobic gas sterilized by a sterilization filter (pore size: 0.2 μm, material: PVDF) was continuously supplied.

The production of equol was confirmed through high-performance liquid chromatography (HPLC) analysis.

### pH Adjustment Step

The main culture solution in which equol production was confirmed was collected in a glass container, the pH was adjusted, and the solution was sterilized through heating. For comparison, a sample was prepared in which sterilization by heating was carried out without adjusting the pH.

In addition, sodium hydroxide was added to the main culture solution to increase the pH to 8, 9, 10, or 11, sterilization by heating was carried out, insoluble components were precipitated by a centrifugal separator in the same manner as in Example 3, and the supernatant was collected. The settled precipitate was subjected as is to isoflavone analysis.

The results are presented in Table 7.

Table 7 shows that when soybean germ was used as the raw material, equol and isoflavones were also present in the precipitate, but by adjusting the pH to the alkaline range in the pH adjustment step, the isoflavone concentration and equol concentration in the precipitate were both decreased, the isoflavone concentration and equol concentration in the supernatant were increased, and the recovery rate of isoflavones was increased.

Since the recovery rate of equol can be increased and isoflavones other than equol can also be expected to have physiological activity, an effect that cannot be achieved by equol alone can be expected by improving the isoflavone concentration.

Furthermore, by removing, through centrifugation, soybean germs that are insoluble in water, a clear aqueous solution can be recovered, and suitability for use in beverages and the like can be increased.

TABLE 7
Filtrate Recovery Amount and Analysis Results

| | | Supernatant | | Precipitate | |
|---|---|---|---|---|---|
| | | Equol | Isoflavone | Equol | Isoflavone |
| pH adjuster | pH | (g/L) | (g/L) | (mg/g) | (mg/g) |
| — | No adjustment | 0.018 | 0.03 | 2.15 | 7.36 |
| NaOH | 8 | 0.038 | 0.10 | 1.72 | 6.37 |
| | 9 | 0.047 | 0.19 | 1.70 | 6.18 |
| | 10 | 0.048 | 0.45 | 1.69 | 2.72 |
| | 11 | 0.092 | 0.61 | 1.49 | 1.66 |

### Example 8

The same procedure as in Example 7 was carried out with the exception that the Asaccharobacter celatus DSM 18785 strain was used instead of the Adlercreutzia equolifaciens DSM 19450 strain that was used in Example 7.

The results are presented in Table 8.

As in Example 7, it was found that when soybean germ was used as the raw material, equol and isoflavones were also present in the precipitate, but by adjusting the pH to the alkaline range in the pH adjustment step, the isoflavone concentration and equol concentration in the precipitate were both decreased, the isoflavone concentration and equol concentration in the supernatant were improved, and the recovery rate of isoflavones was increased.

Since the recovery rate of equol can be increased and isoflavones other than equol can also be expected to have physiological activity, an effect that cannot be achieved by equol alone can be expected by improving the isoflavone concentration.

Furthermore, by removing, through centrifugation, soybean germs that are insoluble in water, a clear aqueous solution can be recovered, and suitability for use in beverages and the like can be increased.

TABLE 8
Filtrate Recovery Amount and Analysis Results

| | | Supernatant | | Precipitate | |
|---|---|---|---|---|---|
| | | Equol | Isoflavone | Equol | Isoflavone |
| pH adjuster | pH | (g/L) | (g/L) | (mg/g) | (mg/g) |
| — | No adjustment | 0.019 | 0.03 | 2.14 | 7.38 |
| NaOH | 8 | 0.039 | 0.11 | 1.72 | 6.41 |
| | 9 | 0.047 | 0.19 | 1.70 | 6.05 |
| | 10 | 0.049 | 0.47 | 1.68 | 2.52 |
| | 11 | 0.093 | 0.60 | 1.48 | 1.76 |

1-13. (canceled)

14. A food composition comprising equol, wherein a pH of the food composition is from 7 to 11.

15. The food composition of claim 14, wherein the food composition is a liquid food composition.

16. The food composition of claim 14, wherein the food composition is a powdered food composition, in which the pH is a pH of the powdered food composition when 50 g of the powdered food composition is dissolved or suspended in 1 L of water.

17. The food composition of claim 14, wherein the food composition comprises at least one hydroxide selected from the group consisting of an alkali metal hydroxide and an alkaline earth metal hydroxide.

18. The food composition of claim 14, wherein the food composition comprises at least one selected from the group consisting of sodium hydroxide, potassium hydroxide, calcium hydroxide, calcium oxide, and magnesium hydroxide.

19. The food composition of claim 14, wherein the food composition comprises a salt of an alkaline earth metal.

20. A method for producing the food composition of claim 14, the method comprising the steps of:
(A) culturing a microorganism that anabolizes at least one equol raw material selected from the group consisting of daidzein glycoside, daidzein, and dihydrodaidzein to produce equol, thereby producing an equol-containing culture solution; and
(B) adjusting a pH of the equol-containing culture solution to a pH from 7 to 11 by adding a pH adjuster to the equol-containing culture solution;
thereby to obtain the food composition.

21. A method for producing the food composition of claim 15, the method comprising the steps of:
(A) culturing a microorganism that anabolizes at least one equol raw material selected from the group consisting of daidzein glycoside, daidzein, and dihydrodaidzein to produce equol, thereby producing an equol-containing culture solution; and
(B) adjusting a pH of the equol-containing culture solution to a pH from 7 to 11 by adding a pH adjuster to the equol-containing culture solution;
thereby to obtain the liquid food composition.

22. A method for producing the food composition of claim 16, the method comprising the steps of:
(A) culturing a microorganism that anabolizes at least one equol raw material selected from the group consisting of daidzein glycoside, daidzein, and dihydrodaidzein to produce equol, thereby producing an equol-containing culture solution;
(B) adjusting a pH of the equol-containing culture solution to a pH from 7 to 11 by adding a pH adjuster to the equol-containing culture solution; and
(C) drying a liquid produced in the step (B);
thereby to obtain the powdered food composition.

23. The method according to claim 20, wherein the pH adjuster includes at least one selected from the group consisting of an alkali metal hydroxide and an alkaline earth metal hydroxide.

24. The method according to claim 20, wherein the pH adjuster includes at least one selected from the group consisting of sodium hydroxide, potassium hydroxide, calcium hydroxide, calcium oxide, and magnesium hydroxide.

25. The method according to claim 20, wherein the pH adjuster includes a salt of an alkaline earth metal.
